(19) United States
(12) Patent Application Publication
(10) Pub. No.: US 2023/0255511 A1
(43) Pub. Date: Aug. 17, 2023

(54) COLLECTION DEVICE COMPRISING A MOUTHPIECE WITH A COMPRESSION TAB

(71) Applicant: ARYBALLE TECHNOLOGIES, GRENOBLE (FR)

(72) Inventors: ROMAIN DUBREUIL, GRENOBLE (FR); KEITH HEWETT, BRISTOL (GB); Saliha SAAD, BRISTOL (GB); ROLLAND GUILLAUME, Grenoble (FR)

(21) Appl. No.: 18/014,149

(22) PCT Filed: Jun. 30, 2021
(86) PCT No.: PCT/EP2021/068105

§ 371 Date: Dec. 31, 2022

(30) Foreign Application Priority Data
Jul. 2, 2020 (FR) FR2006978

(51) Int. Cl. A61B 5/097

(52) U.S. Cl. CPC A61B 5/097; A61B 2560/0425; A61B 2562/16

(57) ABSTRACT

The invention relates to a device (2) for collecting a breath sample from a user and containing target compounds intended to be characterized by an analysis device (3), comprising: a piece (10) for collecting the breath sample comprising a first tab (15) referred to as the compression tab; and a sampling tube (4), intended to be connected to the analysis device (3), passing through an inner pipe (13) of the piece (10), and extending along the longitudinal axis away from and opposite the compression tab (15).

### TECHNICAL FIELD

The field of the invention is that of the collection of a breath sample that may contain analytes to be characterized, in particular on a human subject. More particularly, the aim of the invention is a collection device comprising a mouthpiece with a compression tab, this tab being intended to be inserted into the oral cavity of a user for collecting a sample of their breath. It applies particularly in the characterization of analytes present in the collected breath sample, by means for example of an electronic nose using surface plasmon resonance imaging technology.

### PRIOR ART

The document EP2906945 describes a device for collecting a breath sample from a subject, to detect and to characterize the presence of target compounds (ammonia for example) present in this breath sample. Such breath collection devices make it possible to provide information drawn from the analysis of a breath sample and make it possible for example to identify and quantify microorganisms present in the mouth of the subject. These devices may also make it possible to detect and control the presence of a particular substance in the breath of the subject. The aforementioned document indicates that the device comprises a breath collection interface that may consist of a mask or of a tube.

Other collection interfaces are known, such as breath collection mouthpieces inserted into the mouth of the subject. Indirect collection may also be performed thanks to a syringe activated by suction in the mouth of the subject, then by transferring the collected sample into the syringe towards a breath collection device that carries out the analysis.

These breath collection mouthpieces generally require a strict implementation that depends on the know-how of a trained practitioner. The analysis results are highly dependent on the correct execution of the collection method. Collections are then not very reproducible from one person to another. They may also be vulnerable to disturbances inherent to the collection conditions, and particularly to the physiochemical conditions relating to the oral cavity of the subject, which may lead to a measurement noise degrading the quality of the collection and therefore of the characterization of target compounds.

Moreover, oropharyngeal pieces exist making it possible to take gas measurements such as the level of CO₂ in the air inhaled and exhaled by a patient then under anaesthetic. These pieces comprise a main opening making it possible for the patient to breathe during the measurement. These pieces are described particularly in the documents WO2013/189763A1 and CN2728435. However, they are not adapted to carry out a breath collection from the patient particularly due to the main opening.

### DESCRIPTION OF THE INVENTION

The objective of the invention is to remedy at least partly the drawbacks of the prior art, and more particularly to propose a collection device with a piece intended to be inserted at least partially into an oral cavity to collect therein a breath sample. The collection device according to the invention makes it possible to improve the characteristics of the collection, for example in terms of reproducibility and/or in terms of measurement noise.

For this, the object of the invention is a device for collecting a breath sample located in the oral cavity of a user and containing target compounds intended to be characterized by an analysis device, comprising:
a mouthpiece for collecting the breath sample, adapted to close the oral cavity of the user, comprising:
   a first face referred to as the external face, intended to be located outside of the oral cavity, and at which an external hole opens;
   a second face referred to as the internal face, intended to be inserted into the oral cavity, opposite the external face along a longitudinal axis A_{L} of the piece, at which an internal hole opens connected to the external hole by an inner pipe;
   a positioning base, comprising the internal face, and upper and lower contact surfaces intended to be in contact with the teeth of the user when they ensure the holding of the mouthpiece in the oral cavity, the upper and lower surfaces being located on either side of the internal hole along an axis referred to as vertical.

According to the invention, the mouthpiece comprises a first tab referred to as the compression tab, intended to come into contact with the tongue of the user, formed of a thin plate oriented orthogonally to the vertical axis and extending from the internal face and from the side of the lower surface of the base along the longitudinal axis A_{L} over a first predefined length L_{lc}. In addition, the collection device comprises a removable sampling tube, intended to be connected to the analysis device and that may be removed from the piece, passing through the inner pipe, and extending along the longitudinal axis A_{L} away from and opposite the compression tab over a distance D less than or equal to the first length Lie, so that the compression tab prevents any contact of the tongue with the sampling tube.

Some preferred but nonlimiting aspects of this collection device are the following.

The collection device may comprise a second tab referred to as the protection tab, formed of a thin plate oriented orthogonally to the vertical axis and extending from the internal face and from the side of the upper surface of the base along the longitudinal axis A_{L} over a first predefined length Lₗₚ greater than or equal to the distance D, away from the sampling tube, and arranged opposite the compression tab along the vertical axis.

The compression tab and the protection tab may have identical shapes and extend symmetrically on either side of the longitudinal axis A_{L}.

The collection device may comprise an abutment disposed on the positioning base, intended to be placed in contact with an entrance of the oral cavity.

The abutment may be formed of a protruding flange facing the positioning base.

The collection device may comprise a gripping appendage extending in the extension of the positioning base along the longitudinal axis A_{L}, the gripping appendage comprising the external face.

The positioning base may have a cylindrical shape, defining two contact surfaces, curved and opposite one another along an axis orthogonal to the longitudinal axis A_{L}.

The collection device may comprise a protruding tubular rim facing the positioning base along the longitudinal axis A_{L}.

The collection device may comprise a plurality of inner pipes each receiving a sampling tube.

The invention also relates to an analysis system, comprising a collection device according to any one of the preceding features, and an analysis device connected to the sampling tube.

The analysis device may comprise a measuring chamber intended to receive the target compounds contained in the breath sample, wherein a plurality of distinct sensitive sites is located each comprising receivers capable of interacting with the target compounds, the analysis device comprising a surface plasmon resonance imaging detector, or comprising a plurality of distinct electromechanical resonators each forming a sensitive site.

The invention also relates to a method of collection by means of the collection device according to any one of the preceding features, wherein the mouthpiece is partially inserted into the oral cavity of the user in view of a breath collection, the compression tab being placed in contact with the tongue of the subject. Moreover, preferably, the user breathes through the nose during the collection of the breath sample, or holds their breath.

### BRIEF DESCRIPTION OF THE DRAWINGS

Other aspects, aims, advantages and features of the invention will become more apparent upon reading the following detailed description of preferred embodiments, given by way of nonlimiting example, and made with reference to the appended drawings wherein:

FIG. 1 is a schematic and partial view, from the side, of an analysis system comprising an analysis device and a collection device according to one embodiment;

FIG. 2 is a schematic view, in longitudinal section and in perspective, of the collection mouthpiece illustrated in FIG. 1;

FIG. 3A is a schematic view, in perspective of the collection mouthpiece illustrated in FIG. 1;

FIG. 3B is a schematic view, according to another perspective, of the collection mouthpiece illustrated in FIG. 1;

FIG. 3C is a schematic view, from the front, of the collection mouthpiece illustrated in FIG. 1;

FIG. 4 is a schematic side view of the collection mouthpiece illustrated in FIG. 1, inserted into the oral cavity of a user.

### DETAILED DESCRIPTION OF PARTICULAR EMBODIMENTS

In the figures and in the remainder of the description, the same references represent identical or similar elements. In addition, the various elements are not shown to scale in such a way as to give preference to the clarity of the figures. Moreover, the various embodiments and variants are not exclusive of one another and may be combined together. Unless otherwise specified, the terms “substantially”, “approximately”, “in the order of” mean to the nearest 10%, preferably to the nearest 5%. Moreover, the terms “between . . . and . . . ” and equivalent mean that the ranges are included, unless otherwise specified.

The invention relates to a device for collecting a breath sample that may contain target compounds to be characterized. This collection device comprises a mouthpiece with a compression tab and a removable sampling tube intended to be connected to a device for analyzing target compounds. The mouthpiece is adapted to close the oral cavity of the user.

Generally, analytes are elements contained in the breath sample and intended to be characterized by the analysis device. They may, by way of illustration, be bacteria, viruses, proteins, lipids, volatile organic molecules, inorganic compounds, etc. They may also concern sulfur compounds present in an oral cavity of a subject (human person, animal, etc.).

The analysis device may be called “electronic nose”. It may use surface plasmon resonance measuring technology (SFR) such as described for example in the publication by Brenet et al. titles Highly-Selective Optoelectronic Nose based on Surface Plasmon Resonance Imaging for Sensing Gas Phase Volatile Organic Compounds, Anal. Chem. 2018, 90, 16, 9879-9887. As a variant, other measuring technologies may be implemented, such as electromagnetic resonator measuring of the MEMS or NEMS type (for example, described in the document EP3184485). More broadly, the measuring device may be of the resistive, piezoelectric, mechanical, acoustic or optical type.

As described in detail later, the piece of the collection device is intended to be partially inserted into the oral cavity, and is adapted to close the oral cavity of the user. It comprises a compression tab intended to come into contact with or facing a surface that delimits the oral cavity, that is to say the inside of the oral cavity. Opposite or facing means to be located perpendicular, along a vertical axis and orthogonal to the longitudinal axis A_{L} of the piece. The axis is referred to as vertical in connection with the teeth biting axis. The sampling tube passes through the piece and its free end intended to collect the breath sample is then located opposite the compression tab. The length of the compression tab is greater than or equal to the distance according to which the sampling tube extends protruding facing an internal face of the piece. Thus, the compression tab makes it possible to prevent a surface that delimits the oral cavity from being able to move and disturbing the collection of the breath sample by the sampling tube. Here, this surface is particularly the tongue of the subject (human person, or animal), which, by moving, during the collection, may modify the local physiochemical conditions in the oral cavity (presence of drops of saliva, higher level of moisture, mechanical interaction with the tube, etc.).

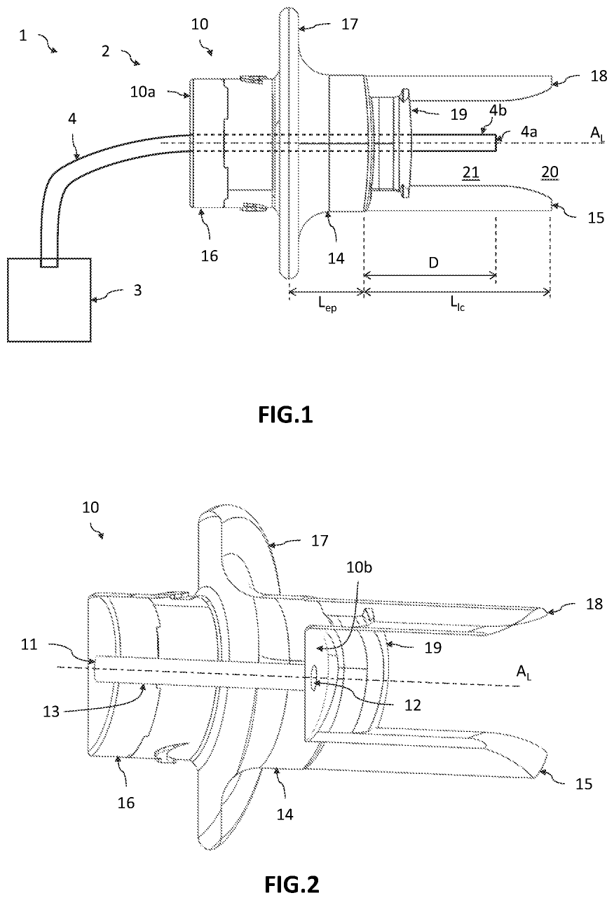

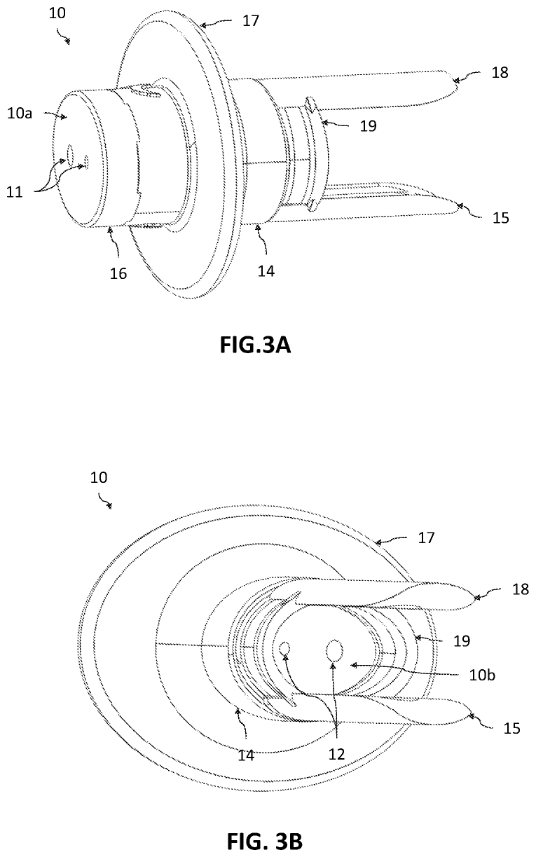

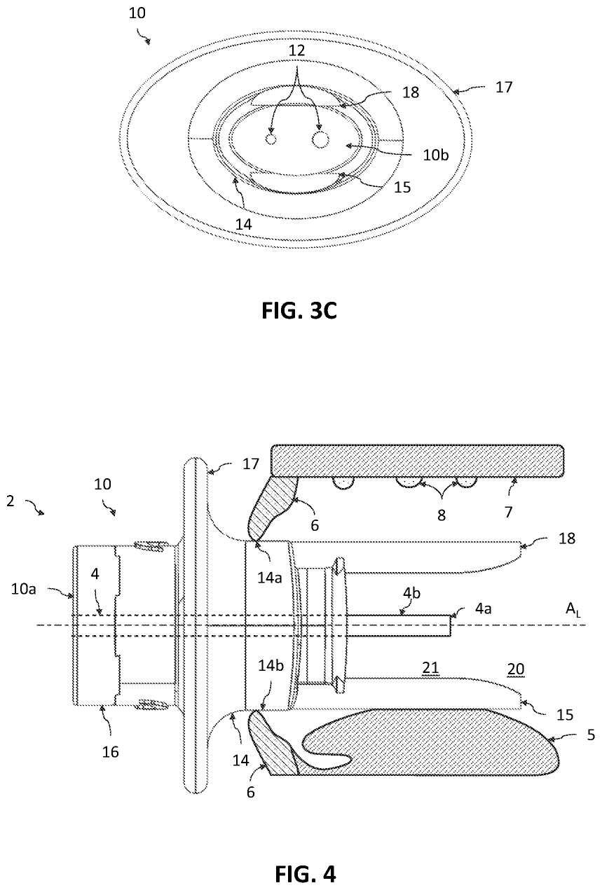

FIG. 1 is a schematic side view of an analysis system 1 comprising a collection device 2 according to one embodiment, fluidly connected to an analysis device 3. FIGS. 2, 3A and 3B illustrate the mouthpiece 10 according to various views. In this example, the collection is carried out in the oral cavity of a human person. Indeed, this concerns collecting a breath sample, this breath sample comprising target sulfur compounds to be characterized, located at a non-zero distance from the entrance of the oral cavity.

The collection device 2 also comprises a collection mouthpiece 10 intended to be partly inserted into the oral cavity, which comprises a first tab 15 referred to as the compression tab, and a removable sampling tube 4 that extends opposite the compression tab 15 and is intended to be connected to the analysis device 3.

The mouthpiece 10 comprises a structure having:
a first face 10a, referred to as the external face since intended to be located outside of the oral cavity, at which a first hole 11 referred to as the external hole opens; and
a second face 10b, referred to as the internal face intended to be located in the oral cavity, opposite the external face 10a along a longitudinal axis A_{L} of the mouthpiece 10, at which a second hole 12 referred to as the internal hole opens connected to the external hole 11 by an inner pipe 13.

The two external 10a and internal faces 10b here are parallel with one another, but they may be inclined in relation to one another. The external face 10a here comprises two external holes 11, and the internal face 10b comprises two internal holes 12, two inner pipes 13 connecting an external hole 11 to an internal hole 12. As a variant, the faces 10a, 10b may comprise a single external hole and only one internal hole, or a plurality of holes. An inner pipe 13 makes a sealed connection between the external hole 11 and internal holes 12. The structure of the mouthpiece 10 is made preferably of a rigid and chemically inert material.

The mouthpiece 10 comprises a positioning base 14, which here forms a portion of the structure of the piece. The base 14 comprises the internal face 10b and is intended to be at least partly inserted into the oral cavity. In this breath collection application, it is intended to be held in the oral cavity by a biting force applied by the teeth of the subject on the base 14 along the vertical axis. More specifically, it has a lower surface 14a and an upper surface 14b, intended to be in contact with the teeth of the user that then ensures the holding of the mouthpiece 10 in the oral cavity. The surfaces 14a and 14b are opposite one another along the vertical axis (axis of biting by the teeth of the user) and are located on either side of the internal hole 12.

It should also be noted that the mouthpiece 10 is adapted to fill the entrance of the oral cavity, even if it does not necessarily concern a sealed filling. For this, the base 14 has transverse dimensions (in a plane orthogonal to the axis A_{L}) at most equal to the dimensions of the entrance of the oral cavity. The flange 17 extends from the base 14 over its entire circumference and has dimensions greater than the entrance of the oral cavity. Thus, during the measurement, the breath remains confined in the oral cavity and is not or little diluted by the external air entering into the oral cavity.

The mouthpiece 10 preferably comprises a gripping appendage 16, making it possible for a practitioner or for the subject themselves to manipulate the mouthpiece 10 to partially insert it into the oral cavity. The appendage 16 comprises the external face 10a and is not intended to be inserted into the oral cavity.

In this example, the appendage 16 and the base 14 are two portions made from one piece of the same structure. As a variant, it may concern two portions assembled to one another but removable. They each have an elliptical or oval-based cylindrical shape, disposed in the extension of one another along the longitudinal axis A_{L} of the mouthpiece. The cylindrical base may have other shapes, such as for example a polygonal, for example rectangular, shape.

The mouthpiece 10 comprises a first tab 15 referred to as compression tab. The compression tab 15 is a thin plate made of rigid material, preferably identical to that of the structure of the mouthpiece 10, which has a length along the longitudinal axis A_{L} greater than its thickness. It is oriented substantially orthogonal to the vertical axis, therefore horizontally and not vertically. In other words, its width is substantially orthogonal to the vertical axis.

It has an outer surface that is intended to come into contact with or facing a surface that delimits the oral cavity, and an inner surface, opposite the outer surface, which is intended to be opposite the sampling tube 4. It extends over at least one portion of the edge of the internal face 10b. It extends from the internal face 10b along the longitudinal axis A_{L} over a predefined length L_{lc}. In this example, it is positioned at the edge of the internal face 10b from the side of the lower surface 14a. The compression tab 15 is intended to be in contact with a surface of the oral cavity, here in contact with the tongue, to thus prevent it from coming into contact with the free end 4a of the sampling tube 4. As mentioned below, its length L_{lc} is greater than or equal to the distance D according to which the sampling tube 4 extends along the longitudinal axis A_{L} from the internal face 10b. This makes it possible to improve the protection of the sampling tube 4 and of its free end 4a facing the surface of the oral cavity, and particularly of the tongue. Thus, the risks of disturbing the operating conditions of the collection are reduced, which makes it possible to improve its repeatability and limits the measurement noise.

The collection device 2 also comprises a removable sampling tube 4, intended to be connected to the analysis device 3, and having one end referred to as the free end 4a intended to be located in the oral cavity. The sampling tube 4 passes through the mouthpiece 10 by the inner pipe 13, and extends along the longitudinal axis A_{L} by being away from and opposite the compression tab 15. In other words, the sampling tube 4, and more specifically its portion referred to as the internal portion 4b that extends from the internal hole 12 in a direction opposite the internal hole 11, extends substantially parallel to the compression tab 15, and is distant (no contact) from it along an axis referred to as the transverse axis that is orthogonal to the longitudinal axis A_{L}. The internal portion 4b of the sampling tube 4 extends along the longitudinal axis A_{L} over a distance D less than or equal to the length L_{lc} of the compression tab 15. Thus, it is possible to prevent the free end 4a of the sampling tube, from which the breath sample is collected, from coming into contact with a surface delimiting the oral cavity. This distance D may be in the order of half or three quarters of the length L_{lc}.

The sampling tube 4 is removable and may therefore be inserted into and removed out of the inner pipe 13. It extends away, over its entire length, facing the compression tab 15 such as facing the protection tab 18. Thus, it is possible, during a series of measurements with the same patient or user, to use the same mouthpiece 10 but a plurality of sampling tubes 4: one tube may be used for one measurement, then removed, and another tube is inserted. Thus, having to clean the mouthpiece 10 between each measurement is prevented, and the removal of the used tube and the insertion of a new tube simplify the measurements.

In addition, the fact that the sampling tube 4 is removable in the inner pipe 13 makes it possible to accurately adjust the position of its end 4a in the oral cavity (that is to say its penetration depth), depending on the target components that it is desired to characterized. Indeed, it is known that the microbial species do not have the same concentration between the front and the back of the oral cavity.

FIG. 4 schematically and partially shows, from the side, the mouthpiece 10 illustrated in FIG. 1 inserted partially into an oral cavity. This is shown in longitudinal section, and the tongue 5, the teeth 6 and the palate 7 are shown schematically.

Here, in the aim of making it possible for the subject to hold the mouthpiece 10 with their teeth 6, the base 14 having two contact surfaces 14a, 14b, opposite one another facing the vertical axis and arranged on either side of the internal hole 12. These contact surfaces 14a, 14b are intended to receive the teeth 6 of the subject, who then applies a biting force towards one another.

The mouthpiece 10 advantageously comprises an abutment 17, here in the form of a flange, disposed on the base 14 and adjacent to the appendage 16, which may constitute a positioning reference facing the entrance of the oral cavity. The abutment 17 here is a wall that extends transversally to the longitudinal axis A_{L}, over at least one portion of the periphery of the base 14. In this example, the abutment 17 is intended to be pressed against the lips of the subject (not shown) when they bite the base 14, so that the appendage 16 and the abutment 17 remain outside of the oral cavity. It contributes to file the entrance of the oral cavity.

Preferably, the mouthpiece 10 comprises a second tab 18, referred to as the protection tab, which here is substantially symmetrical to the compression tab 15 facing the longitudinal axis A_{L} (it is therefore opposite the tab 15 along the vertical axis). It is a thin plate made of rigid material, preferably identical to that of the structure of the mouthpiece 10, which has a length along the longitudinal axis A_{L} greater than its thickness. It has an outer surface that is intended to come into contact with or facing a surface that delimits the oral cavity, and an inner surface, opposite the outer surface, which is intended to be opposite the sampling tube 4 and the inner surface of the compression tab 15. It is oriented orthogonally to the vertical axis, therefore is not horizontal.

It extends over at least one other portion of the edge of the internal face 10b, from the side of the upper surface 14b. It also extends from the internal face 10b along the longitudinal axis A_{L} over a predefined length Lₗₚ. In this example, it is also positioned at the edge of the internal face 10b. The protection tab 18 is intended to be at least facing a surface of the oral cavity, here facing the palate, to thus prevent any drops 8 from falling on the internal portion 4b of the sampling tube 4, and more specifically from falling on the free end 4a. It therefore extends away from the sampling tube 4. Such drops could thus increase the level of moisture of the breath sample collected, which may induce a measurement noise during the characterization of target compounds. Its length Lₗₚ is greater than or equal to the distance D according to which the sampling tube 4 extends along the longitudinal axis A_{L} from the internal face 10b.

The compression 16 and protection tabs 18 here are distinct from one another. They delimit an internal space wherein the internal portion 4b of the sampling tube 4 is located. This internal space has a back opening 20 oriented towards the longitudinal axis A_{L} and whereby the breath sample is suctioned. Here, it has two lateral openings 21, opposite one another along a transverse axis. As a variant, the two tabs may be joined laterally via lateral walls, and thus form the same peripheral wall that extends continuously around the internal portion 4b of the sampling tube 4. In this case, this peripheral compression and protection wall advantageously has a plurality of through holes (grooving perforation, etc.), arranged at a transverse axis referred to as the horizontal axis, that is to say orthogonal to the longitudinal axis A_{L} and to the vertical axis (which is oriented from the tongue 5 to the palate 7).

As mentioned above, the mouthpiece 10 comprises at least one inner pipe 13, with corresponding external 11 and internal holes 12, to make it possible to insert the sampling tube 4 and therefore to collect the breath sample. In the present example, the mouthpiece 10 comprises two inner pipes 13 making it possible to insert two tubes and therefore to perform two simultaneous breath collections. As a variant, the mouthpiece 10 comprises only one inner pipe 13, or as many inner pipes 13 as desired simultaneous collections. The tubes may have different penetration depths, to measure various target compounds or a concentration gradient of the same target compound. An inner pipe 13 has dimensions adapted to those of the sampling tube 4 to make it possible to insert and remove the tube while limiting fluidic exchanges between the external air and the breath located in the oral cavity.

The mouthpiece here comprises a tubular rim 19 extending the cylindrical profile of the base 14 and therefore extending protruding facing the base 14 along the longitudinal axis A_{L}, the internal holes 12 opening in the space delimited by this tubular rim 19.

The mouthpiece 10 is preferably made of only one part, for example by injection molding in a resin adapted to current sterilization methods. The base 14 is solid (without main opening, except the inner pipe(s) to ensure the passage of the sampling tube(s)). Its constitution with a compression tab 15 and a protection tab 18 identical to one another, extending symmetrically along the longitudinal axis A_{L}, simplifies the use by reducing the instructions to the single directive of placing one of the two elements against the tongue of the subject.

An example of collection method is now described within the scope of collecting the breath of a subject, by means of the collection device described with reference to FIG. 1.

The mouthpiece 10 is firstly sterilized by any known method, for example by steam in an autoclave device. It is then optionally cleaned to eliminate any trace of sterilization product that could interfere with the breath collection.

According to a particularly advantageous embodiment, illustrated in FIGS. 1 and 4, the mouthpiece 10 is firstly equipped with at least one removable and disposable sampling tube 4. The sampling tube 4 is inserted into the inner pipe 13 and thus passes through the base 14 and the appendage 16, by protruding on the one hand through the external hole 11 and on the other hand through the internal hole 12. From the internal hole 12, the tube 4 extends along the longitudinal axis A_{L} up to a calibrated distance D defined between the internal face 10b and the free end 4a of the tube 4.

The end opposite the sampling tube 4 is connected to an analysis device 3 adapted to perform the collection by suction, the detection and the characterization of target compounds present in the breath sample.

Once the sampling tube 4 is in place, the gripping appendage 14 is grasped by the subject, and the mouthpiece 10 is inserted into the mouth, so that the compression tab 15 comes against the tongue 5 of the subject who subsequently bites with their teeth 6 on the base 14 by placing their lips in contact with the abutment 17. The entrance of the oral cavity is then filled. The analysis device 3 is then activated to perform the breath collection by suction then the characterization of target compounds.

During the breath collection, the breath sample is suctioned by the free end of the 4a of the sampling tube 4, that is to say at a calibrated longitudinal distance D inside the mouth of the subject. This distance D is selected depending on the exact area of collection desired, which may require a sampling, for example, rather close to the teeth or rather towards the back of the oral cavity. The collection is preferably carried out towards the back of the oral cavity insofar as it appears that the concentration in sulfur compounds to be characterized is higher there. It should be noted that during the collection, the subject breathes through the nose and cannot breathe through the mouth: the mouthpiece 10 fills the entrance of the oral cavity, and the dimensions of the inner pipe 13 like those of the tube 4 are not enough to make it possible for the subject to breathe through the mouth or to make it possible for a fluidic exchange between the breath and the external air. Thus, the breath is prevented from becoming diluted in the pulmonary air flow, so that the concentration of target compounds to be characterized is not degraded.

During the collection, the compression tab 15 prevents the tongue 5 of the subject from coming into contact with the sampling tube 4 and thus prevents the saliva of the subject from disturbing the breath collection, for example by introducing into the collected breath vapor from the evaporation of saliva close to the free end 4a. Likewise, the protection tab 18 protects the sampling tube 4, and its free end 4a, from the falling of saliva droplets 8, for example, from the palate 7 of the subject, so as not to disturb the breath collection.

When the mouthpiece 10 comprises for example two inner pipes 13 that are equipped with two sampling tubes 4, the inner pipes 13 may have different sections, and therefore house sampling tubes 4 of different sections or diameters. These sampling tubes 4 may be connected to the same analysis device, or to two different analysis devices each performing a different analysis of its respective collection. They may have different penetration depths.

Two simultaneous breath collections may be performed with their respective section for the sampling tubes 4 as well as their respective calibrated distance D. Two simultaneous collections with sampling tubes 4 positioned according to two different calibrated distances D make it possible to perform two collections at two different depths in the oral cavity.

Alternative embodiments of the mouthpiece 10 may be implemented without departing from the scope of the invention.

Thus, the base 14 may comprise a protruding duct facing the internal face 10b along the axis A_{L} and opening in the internal hole 12, ensuring an additional mechanical holding of the internal portion 4b of the sampling tube 4.

Moreover, the abutment 17 may moreover be provided to come directly against the teeth of the subject rather than against their lips. The abutment 17 may then be formed by a groove made in the base 14, or by a flange of adapted diameter.

Particular embodiments have just been described. Various variants and modifications will become apparent to the person skilled in the art.

1-13. (canceled)

14. A collection device for collecting a breath sample from a user and a containing target compound to be characterized, comprising:
a mouthpiece for collecting the breath sample, configured to close an oral cavity of a user, and comprising:
an external face, intended to be located outside of the oral cavity, comprising an external hole opening;
an internal face, intended to be inserted into the oral cavity, opposite the external face along a longitudinal axis A_{L} of the mouthpiece, comprising an internal hole opening connected to the external hole by an inner pipe;
a positioning base, comprising the internal face, and comprising an upper contact surface and a lower contact surface configured to be in contact with teeth of the user when the user holds the mouthpiece in the oral cavity, the upper contact surface and the lower contact surface being located on either side of the internal hole, along a vertical axis;
wherein the mouthpiece comprises a compression tab, configured to contact the user's tongue, the compression tab comprising a thin plate oriented orthogonally to the vertical axis and extending from the internal face and from a side of the lower contact surface of the positioning base along the longitudinal axis A_{L} over a first predefined length L_{lc}; and
wherein the collection device comprises a removable sampling tube, configured to be connected to an analysis device, the removable sampling tube passing through the inner pipe, and extending along the longitudinal axis A_{L} away from and opposite the compression tab over a distance D less than or equal to the first length L_{lc}, so that the compression tab prevents any contact of the user's tongue with the removable sampling tube.

15. The collection device of claim 14, comprising a protection tab, comprising a thin plate oriented orthogonally to the vertical axis and extending from the internal face and from a side of the upper contact surface of the positioning base along the longitudinal axis A_{L} over a first predefined length Lₗₚ greater than or equal to the distance D, away from the removable sampling tube, and arranged opposite the compression tab along the vertical axis.

16. The collection device of claim 15, wherein the compression tab and the protection tab have identical shapes and extend symmetrically on either side of the longitudinal axis A_{L}.

17. The collection device of claim 14, comprising an abutment on the positioning base, configured to be in contact with an entrance of the oral cavity.

18. The collection device of claim 17, wherein the abutment comprises a protruding flange facing the positioning base.

19. The collection device of claim 14, comprising a gripping appendage extending in an extension of the positioning base along the longitudinal axis A_{L}, the gripping appendage comprising the external face.

20. The collection device of claim 14, wherein the positioning base has a cylindrical shape, defining two contact surfaces, curved and opposite one another along an axis orthogonal to the longitudinal axis A_{L}.

21. The collection device of claim 14, comprising a protruding tubular rim facing the positioning base along the longitudinal axis A_{L}.

22. The collection device of claim 14, comprising a plurality of inner pipes each receiving a removable sampling tube.

23. An analysis system, comprising the collection device of claim 14 and further comprising an analysis device connected to the removable sampling tube.

24. The analysis system of claim 23, wherein the analysis device comprises a measuring chamber configured to receive a target compound contained in the breath sample, the measuring chamber comprising a plurality of distinct sensitive sites each sensitive site comprising a receiver configured to interact with the target compound, the analysis device comprising a surface plasmon resonance imaging detector.

25. The analysis system of claim 23, wherein the analysis device comprises a measuring chamber configured to receive a target compound contained in the breath sample, the measuring chamber comprising a plurality of distinct sensitive sites each sensitive comprising a distinct electromechanical resonator.

26. A method comprising a step of collecting a breath sample by means of the collection device of claim 14, wherein the mouthpiece is partially inserted into an oral cavity of a user, the compression tab being in contact with the user's tongue.

27. The method of claim 26, wherein, during the step of collecting the breath sample, the user breathes through his nose.
